(19) United States
(12) Patent Application Publication
(10) Pub. No.: US 2006/0052656 A1
(43) Pub. Date: Mar. 9, 2006

(54) Implantable devices using magnetic guidance

(75) Inventors: Mariam N. Maghribi, Livermore, CA (US); Peter A. Krulevitch, Pleasanton, CA (US); James Courtney Davidson, Livermore, CA (US); Julie K. Hamilton, Tracy, CA (US)

Correspondence Address: Eddie E. Scott;Assistant Laboratory Counsel, Lawrence Livermore National Laboratory, P.O. Box 808, L-703, Livermore, CA, 94551, US

(73) Assignee: The Regents of the University of California

(21) Appl. No.: 10/938,816

(22) Filed: Sep. 9, 2004

(51) Int. Cl. A61N 2/00

(52) U.S. Cl. 600/12

(57) ABSTRACT

A system for movement in a body and performing a function on the body. The system comprises a polymer body portion, an electronic unit carried by the polymer body portion, a circuit in the polymer body portion operatively connected to the electronic unit, pieces in the polymer body portion, and a system for controlling movement of the polymer body portion utilizing magnetic forces controlling movement of the pieces. The system utilizes magnetic guidance to control movement of a polymer body portion.

The United States Government has rights in this invention pursuant to Contract No. W-7405-ENG-48 between the United States Department of Energy and the University of California for the operation of Lawrence Livermore National Laboratory.

### BACKGROUND

1. Field of Endeavor

The present invention relates to magnetic guidance and more particularly to a system utilizing magnetic forces to control movement of a polymer body portion.

2. State of Technology

U.S. Pat. No. 6,475,233 issued Nov. 5, 2002 to Peter R. Werp et al for a method and apparatus for magnetically controlling motion direction of a mechanically pushed catheter provides the following state of technology information, “There is a large body of conventional (nonmagnetic) stereotactic prior art, in which a frame (e.g., a so-called “BRW Frame”) is attached to the skull to provide a navigation framework. Such a frame has arcs to determine an angle of an “insertion guide” which is usually a straight tube through which some medical therapeutic agent is passed, such as a biopsy tool. These methods have been confined to straightline approaches to a target. There is also a smaller body of prior art in which a handheld permanent magnet or an electromagnet is used to move a metallic implant. Previous implants for delivering medication or therapy to body tissues, and particularly brain tissue, have generally relied upon the navigation of tethered implants within vessels, or navigation of tethered or untethered implants moved intraparenchymally (in general brain tissue) by magnetic force.”

European Patent No. EP 0 422 689 (A2) to Mountpelier Investments, S.A. published Apr. 17, 1991 provides the following state of technology information, “A disclosed embodiment of catheter has magnetically responsive structure at its tip, and the auxiliary device has an electromagnet which is cooperatively associated with the magnetic tip. The auxiliary device is operated to maintain electromagnetic attraction of the magnetic tip structure during an initial phase of the placement process. The auxiliary device is relatively stiffer than the catheter and is used to force the catheter tip past the pylorus when the catheter is introduced into the intestines from the stomach. Thereafter a separate external magnet is used to attract the catheter tip, the electromagnet is de-energised and the auxiliary device withdrawn. Final placement of the catheter is attained through manipulation of the external magnet.”

U.S. Patent Application No. 2004/0006301 by Jonathan C. Sell and Roger N. Hastings for a magnetically guided myocardial treatment system published Jan. 8, 2004 provides the following state of technology information, “A magnetically navigable and controllable catheter device is deployed at the heart wall and this device tunnels into the myocardium. Any of a variety of canalization techniques can be used to tunnel into the heart wall causing mechanical disruption of the tissues, including mechanical needles and RF energy sources as well as direct laser and heated tips. In a preferred embodiment, the catheter device guided by externally applied magnetic fields that are created by a magnetic surgery system (MSS). The MSS applies magnetic fields and gradients from outside the body to manipulate and direct medical devices within the body. The catheter devices of some embodiments of the present invention include magnetic elements that respond to the MSS field or gradient. In general, the physician interacts with a workstation that is associated with the MSS. The physician may define paths and monitor the progress of a procedure. Fully automatic and fully manual methods are operable with the invention.”

### SUMMARY

Features and advantages of the present invention will become apparent from the following description. Applicants are providing this description, which includes drawings and examples of specific embodiments, to give a broad representation of the invention. Various changes and modifications within the spirit and scope of the invention will become apparent to those skilled in the art from this description and by practice of the invention. The scope of the invention is not intended to be limited to the particular forms disclosed and the invention covers all modifications, equivalents, and alternatives falling within the spirit and scope of the invention as defined by the claims.

The present invention provides a system for movement in a body and performing a function on the body. The system comprises a polymer body portion, an electronic unit carried by the polymer body portion, a circuit in the polymer body portion operatively connected to the electronic unit, pieces in the polymer body portion, and a system for controlling movement of the polymer body portion utilizing magnetic forces controlling movement of the pieces. The present invention utilizes magnetic guidance to control movement of a polymer body portion. The system has many uses. For example, the system has use as an implantable biological interface device for artificial stimulation such as retinal, cochlear, and cortical prosthesis.

The invention is susceptible to modifications and alternative forms. Specific embodiments are shown by way of example. It is to be understood that the invention is not limited to the particular forms disclosed. The invention covers all modifications, equivalents, and alternatives falling within the spirit and scope of the invention as defined by the claims.

### BRIEF DESCRIPTION OF THE DRAWINGS

The accompanying drawings, which are incorporated into and constitute a part of the specification, illustrate specific embodiments of the invention and, together with the general description of the invention given above, and the detailed description of the specific embodiments, serve to explain the principles of the invention.

FIG. 1 illustrates a system for movement in a body and performing a function on the body.

FIG. 2 illustrates a cochlear implant system wherein a polymer body portion is inserted in the spiral snail-like cochlea structure by magnetic guidance.

FIG. 3 illustrates a retinal implant system wherein a polymer body portion is position against the retina by magnetic guidance.

FIG. 4 show additional details of the retinal implant system illustrated in FIG. 3.

### DETAILED DESCRIPTION OF THE INVENTION

Referring now to the drawings, to the following detailed description, and to incorporated materials, detailed information about the invention is provided including the description of specific embodiments. The detailed description serves to explain the principles of the invention. The invention is susceptible to modifications and alternative forms. The invention is not limited to the particular forms disclosed. The invention covers all modifications, equivalents, and alternatives falling within the spirit and scope of the invention as defined by the claims.

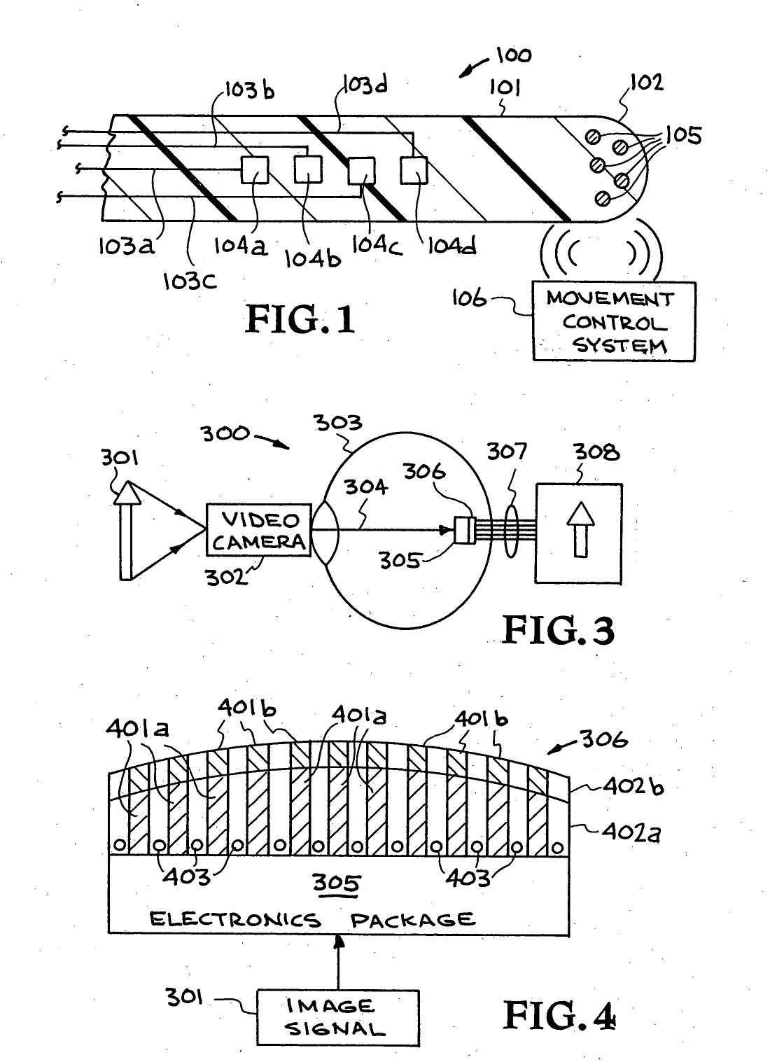

Referring now to in FIG. 1, one embodiment of a system for movement in a body and performing a function on the body is illustrated. The system is generally designated by the reference numeral 100. The system 100 has many uses. For example, the system 100 has uses as implantable biological interface devices for artificial stimulation such as retinal, cochlear, and cortical prosthesis. The system 100 also has uses as precision maneuvering in confined spaces for assembling, modifying or repairing devices such as weapons.

The system 100 comprises a polymer body portion; at least one electronic unit carried by the polymer body portion; a circuit in the polymer body portion operatively connected to the electronic unit; pieces in the polymer body portion; and a system for controlling movement of the polymer body portion utilizing magnetic forces controlling movement of the pieces. The polymer body portion is an elongated body 101 that can be used as an implant or other device. At least one electronic unit is carried by the polymer body portion 101. The electronic units 104A, 104B, 104C, and 104D are carried by the polymer body portion 101. The electronic units 104A, 104B, 104C, and 104D can be electrodes or other electronic units. Interconnected traces 103A, 103B, 103C, and 103D in the polymer body portion 101 operatively connected to the electronic units 104A, 104B, 104C, and 104D respectively.

The elongated polymer body 101 has tip 102 that contains embedded or adhered to pieces 105. The pieces 105 in the tip 102 polymer body portion 101 can be magnetic pieces or non-magnetic pieces. A system 106 controls movement of the polymer body portion 101 utilizing magnetic forces controlling movement of the pieces 105. An external magnet in the system 106 can be used to control movement of the pieces 105 to guide the polymer body portion 101 along a desired path. The pieces 105 can be either magnetic pieces or non-magnetic pieces, or both.

The system 100 is produced using a number of processing steps. The system 100 provides a process for depositing metal features on poly(dimethylsiloxane) which is a type of silicone rubber. With the process Applicants are capable of fabricating stretchable metal traces on PDMS (silicone) using a cost effective batch fabrication process. Applicants have demonstrated selective passivation of these metal traces with PDMS exposing the traces only in areas needed to make contact with the outside world. The embodiment includes improvements in the process of metalizing PDMS, selective passivation, using batch fabrication photolithographic techniques to fabricate PDMS, and producing stretchable metal traces that are capable of withstanding strains of 7% with S.D. 1.

The system 100 provides electrodes 104A-104D contained in a polymer substrate 101. The substrate 101 is composed of a polymer. The polymer is flexible and has the ability to conform to various shapes. The polymer in the embodiment 100 is poly(dimethylsiloxane) (PDMS). Metal traces 103A-103C are provided for electrical connection. The metal traces in the embodiment 100 are composed of lead metal.

The basic process steps for depositing lead and electrode metal on PDMS and subsequent passivation with PDMS will be described. The process is designated generally by the reference numeral 100. The system 100 includes the steps of casting, spon, metallization, spin coating, and release. The applicants approach is to use PDMS as the substrate material to batch produce a low-cost device that is ready for implantation without the need for additional packaging steps. Because PDMS has not previously been used in this type of micromachining application, applicants developed new fabrication processes enabling PDMS patterning, metalization, and selective passivation. The metal features are embedded (deposited) within a thin substrate fabricated using poly(dimethylsiloxane) (PDMS), an inert biocompatible elastomeric material that has simultaneously low water and high oxygen permeability. The conformable nature of PDMS is critical for ensuring uniform contact with the curved surfaces.

PDMS is a form of silicone rubber, a material that is used in many implants and has been demonstrated to withstand the body's chemical and physical conditions without causing adverse side effects and is a favorable material to implant within the body. Robustness of the metalized PDMS is another important design criterion that applicants considered, as stretching and bending occur during fabrication and implantation of the device. The PDMS metalization process was demonstrated to produce devices that will be sufficiently rugged for implantation, with a demonstrated strain to failure of 7%, (SD=1). Applicants attribute the stretchability to a tensile residual stress from curing the PDMS.

In one of the initial steps, silicone is spun onto a silicon handle wafer. The silicone is poly(dimethylsiloxane) known as PDMS. PDMS has very low water permeability and protects the electronic components from the environment. PDMS is flexible and will conform to curved surfaces. It is transparent, stretchable, resinous, rubbery, stable in high temperatures and provides numerous applications for the electronic devices produced by the method.

The silicon handle wafer provides a temporary base for production of the electronic device. Silicon wafers are convenient for the handle material because they are flat, stable, routinely used in microfabrication applications, and they are readily available. However, other materials such as glass, plastic, or ceramic could be used as well. The electronic devices will eventually need to be removed from the handle wafer. Since the flexible polymer layer would become permanently bonded to the surface of the silicon handle wafer, a non-stick layer is first provided on the silicon handle wafer. The step comprises the deposition of gold (or platinum) onto the handle wafer. This allows for removal of the PDMS from the substrate after processing. The gold film facilitates removal of the polymer membrane from the wafer after completion of the fabrication process. Some area on the silicon wafer is left without the gold coating to prevent the PDMS membrane from lifting off during processing, for example, a 2 mm wide ring at the edge is left uncoated with gold. PDMS is then spun onto the wafer at a desired thickness and cured. For example the PDMS may be cured at 66° C. for 24-48 hours (or at manufacturers' specifications).

In a subsequent step the process of forming the electrical circuit lines and the central electrode array of the system 100 is initiated. A photoresist (AZ®1518, Clariant) is spun onto the PDMS membrane surface at 1000 rpm for 20 seconds and baked at 60° C. for 45 minutes. The temperature is brought down slowly (30 min to ramp temperature down) to room temperature to avoid cracking in the photoresist. Prior to photoresist application, the wafer is placed in an oxygen plasma to activate the surface. This allows the resist to wet the PDMS surface preventing beading and ensuring the formation of a smooth and uniform coat of photoresist on the polymer surface. The substrate is placed in the oxygen plasma for 1 minute at an RF power of 100 Watts with oxygen flowing at 300 sccm. The photoresist features are then UV exposed at 279 mJ and developed in AZ developer mixed 1:1 with water for 70 sec. Then the wafer is rinsed under a gentle stream of water and dried using N2. The wafer is placed for a second time in the oxygen plasma to activate the newly exposed PDMS surface, and promote adhesion of the metal, which is deposited in the next step.

In the next step a 150 nm gold film is e-beam evaporated onto the wafer using titanium as the adhesion layer. The e-beam needs to be sufficiently cooled down before removing the parts. Cool down is conducted for 10 min. under vacuum and for 20 min with the system vented, but not open. The metal adheres to the PDMS surface in regions where the photoresist was removed, and the excess metal is removed through a lift-off process by placing the wafer in acetone. The wafer is then prepared for the next step by rinsing with ethanol and drying gently. If the PDMS surface is contaminated or aged, it can be refreshed by soaking in a 20% solution of HCl for 8 min.

In the next steps the process of forming the vias through a passivating layer of PDMS to connect the electrical circuit lines to the electronic components is initiated. A thick photoresist is spun onto the PDMS membrane surface. The photoresist is patterned by exposing the resist to UV through a photomask and developing. The passivating layer of silicone is spun onto the wafer, over the patterned photoresist. The surface is gently swabbed to remove excess PDMS from the top of the photoresist features before stripping the resist. This ensures the removal of the photoresist and the complete clearance of the vias. To strip the resist the wafer is soaked in acetone for 15 min. and then soaked in isoproponol for 5 min. and then rinsed with isoproponol and dried.

Another way of patterning and passivating the PDMS is using a shadow-mask, which is a stencil-like mask exposing the areas that need to be passivated or patterned. A third way of passivating the PDMS is by protecting the areas needed for electrical connection and dipping the wafer in PDMS and curing.

In the next step conductive material is applied to the vias. The vias can be filled with electroplating, conductive silicone adhesive, conductive ink or solder paste. An automated dispenser or applicator machine is used to deposit precise amounts of material in the vias locations. Alternatively, the conductive material can be screen-printed using conductive inks, or liquid ink can be injected into channels formed in the first PDMS layer. As another option, metal can be electroplated in the PDMS vias to form an array of electrical contacts.

In the next step, the surface of the second PDMS layer is rinsed with ethanol and exposed to an oxygen plasma. This activates the surface in preparation for bonding the electronic components to the PDMS. The following step is performed in a nitrogen environment in order to extend the lifetime of the activated surface.

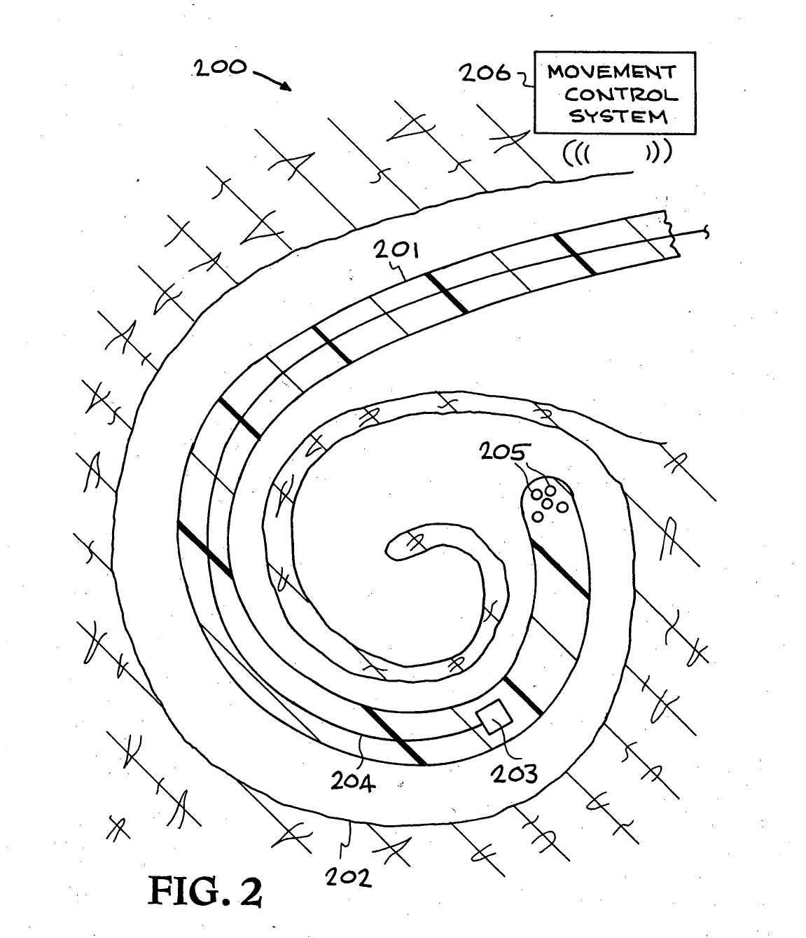

Referring now to FIG. 2, another embodiment of a system for movement in a body and performing a function on the body is illustrated. The system is generally designated by the reference numeral 200. The system 200 provides an implantable biological interface device. In particular the system 200 provides a cochlear prosthesis.

Electrical stimulation devices that substitute for malfunctioning sensory neural structures are important bioengineering applications that require integrating microelectronic systems with biological systems. The use of electrical stimulation to recover lost bodily functions has been pursued for over a century; however, the technology necessary to create an implantable electrical stimulation system has been in existence only for a few decades. Hearing impairment is a chronic condition affecting over 22 million Americans. Medical technologies, such as the cochlear implant, an electronic device designed to provide useful hearing and improved communication ability to people who are profoundly hearing impaired. Cochlear implants bypass damaged hair cells and directly stimulate the hearing nerves with electrical current, enabling people who are profoundly deaf to have some functional hearing. The physical characteristic of the cochlea is spiral snail-like structure hindering device insertion. Special surgical techniques and tools are required in order to minimize trauma and maximize electrode-cochlea interaction. The present invention provides an alternative to current methods of insertion in that the present invention uses embedded or attached material with magnetic guidance.

The cochlear implant system 200 comprises a polymer body portion 201 that is inserted in the spiral snail-like cochlea structure 202. At least one electronic unit 203 is carried by the polymer body portion 201. A circuit 204 in the polymer body portion operatively connected to the electronic unit 203. Pieces 205 in the polymer body portion 201 and a system 206 for controlling movement of the polymer body portion 201 utilizing magnetic forces guides the cochlear implant polymer body portion 201 through the spiral snail-like cochlea structure 202.

The elongated polymer body 201 tip contains the embedded or adhered to pieces 205. The pieces 205 in the tip polymer body portion 201 can be magnetic pieces or non-magnetic pieces. The system 206 controls movement of the polymer body portion 201 utilizing magnetic forces controlling movement of the pieces 205. An external magnet in the system 206 can be used to control movement of the pieces 205 to guide the polymer body portion 201 along a desired path through the spiral snail-like cochlea structure 202. The pieces 205 can be either magnetic pieces or non-magnetic pieces, or both.

The cochlear implant system 200, and in particular the polymer body portion 201, is batch fabricated using a silicone-based technology. The silicone-based technology is described in U.S. Patent Application No. 2003/0097166 by Peter Krulevitch, Dennis L. Polla, Mariam Maghribi, and Julie Hamilton for a Flexible Electrode Array for Artificial Vision published May 22, 2003; U.S. Patent Application No. 2003/0097165 by Peter Krulevitch, Dennis L. Polla, Mariam Maghribi, Julie Hamilton, and Mark S. Humayun for a Flexible Electrode Array for Artificial Vision published May 22, 2003; and U.S. Patent Application No. 2004/0018297 by Courtney Davidson, Peter Krulevitch, Mariam Maghribi, William Benett, Julie Hamilton, and Armando Tovar for Conductive Inks for Metalization in Integrated Polymer Microsystems published Jan. 29, 2004. U.S. Patent Applications Nos. 2003/0097166, 2003/0097165, and 2004/0018297 are incorporated herein in their entirety by this reference.

In one embodiment, of the cochlear implant system 200, the tip of the device to be implanted is embedded or adhered to with magnetic material 205. An external magnet in the system 206 is used to guide the polymer body portion 201 along the spiral path of the cochlea 202. In one embodiment the system 206 includes an electromagnet. In another embodiment the system 206 includes a permanent magnet.

Once implanted, the tip can be removed if desired so that the magnetic material does not remain in the tissue. The implantable device is batch fabricated using a silicone-based technology and the magnetic particles 205 are patterned and doped into the polymer body portion 201 prior to curing or following the curing process magnetic material is attached to the polymer body portion 201 using biocompatible adhesives.

Referring now to FIG. 3, a retinal prosthesis incorporating a system of the present invention is illustrated. This is an embodiment of the present invention that provides a system that restores vision to people with certain types of eye disorders. The system is generally designated by the reference numeral 300.

The human eye is roughly spherical with an axial length of approximately 24 mm on average. There are three main compartments of the eye. The outermost compartment is called the anterior chamber; it is comprised of the cornea in the front and the iris or colored section on the inside. The cornea is a clear convex window that is integrated onto the main sphere of the eye comprised of the sclera, which is the white region of the eye. The junction of where the cornea meets the sclera is the limbus. The second compartment is the cavity immediately behind the iris and in front of the lens, and is called the posterior chamber. Both the anterior and posterior chambers are filled with a watery liquid, called aqueous humor, which percolates through the eye, providing nourishment and cleansing. The third and largest compartment is the cavity of the vitreous. A gelatinous substance, called the vitreous humor, occupies this space and maintains the structure of the eye.

The eye is a complex optical system and the ability to see is dependent on the actions of several structures in and around the eyeball. When one looks at an object, light rays are reflected from the object to the cornea. From there, the light travels through clear aqueous fluid, and passes through a small aperture called the pupil. As muscles in the iris relax or constrict, the pupil changes size to adjust the amount of light entering the eye. Light rays are focused through the crystalline lens. The sharpness of the final picture is adjusted automatically by changing the shape, and thereby the refractivity, of the crystalline lens. This focusing system is so powerful that the light rays intersect at a point just behind the lens inside the vitreous humor and diverge from that point back to the retina. The resulting image on the retina is upside-down. Here at the retina, the light rays are converted to electrical impulses. These impulses are then transmitted through the optic nerve, an electrochemical conduit, connecting the eyeball to the brain. From there, optic radiations extend to the visual cortex in the occipital lobe of the brain toward the visual cortex where the image is translated and perceived in an upright position.

Like a camera, the eye consists of a lens and a recording medium to produce an image. The eyeball's lens is compromised of the cornea, crystalline lens, and vitreous to refract and focus the light, and a film consisting of the retina on which the rays are focused. If any one or more of the lens components is not functioning properly, the result is refractive problems and poor focusing of images on the retina. If the retina is not functioning properly then more dramatic consequences may result, including blindness.

The retina is a thin (0.25 mm) delicate neural tissue, which senses the light entering the eye. It converts this light information into neural electrical signals. It is the innermost layer lining the inside of the eye opposite the crystalline lens and is comprised of hundreds of millions of nerves distributed within its layers, which also contain the vessels and photochemical receptors necessary for vision. The retina is composed of approximately 126 million photoreceptors and one million ganglion cells. The axons of the ganglion cells form the optic nerve, which extends into the visual cortex of the brain. There are many millions of interneurons packed into the retina intervening between the photoreceptors and the ganglion cells. Signal processing and image convergence is performed in all neural cell layers involving bipolar cells, horizontal cells, amacrine cells and ganglion cells.

Referring again to FIG. 3, a video camera 302 captures an image 301. The image 301 is sent by a cable connection, a laser or RF signal 304 into a patient's eye 303. An electronics package 305 within the eye 303 receives the image signal 301 and send it to an electrode array 306. The electrode array 306 comprises a substrate made of a compliant material with electrodes and conductive leads embedded in the substrate. The electrodes contact tissue of the retina within the eye 303. The electrode array 306 stimulates retinal neurons 307. The retinal neurons 307 transmit the signal 301 to be decoded in the brain 308. The image signal 301 is sent to the electrode array 306. The electrode array 306 is connected to the retina and the electrical stimulus is sent to the ganglion cells based on information received from the external camera.

Referring now to FIG. 4, additional details of the retinal prosthesis 300 illustrated in FIG. 3 are shown. The electronics package 305 transmits the signal 301 to the electrode array 306. The electrode array 306 has microstimulator electrodes 401A and 401B connected to a conformable PDMS substrate 402A and 402B. Examples of the conformable PDMS substrate 402A and 402B, the production of the conformable PDMS substrate 402A and 402B, and the retinal prosthesis 300 are described in U.S. Patent Application No. 2003/0097166 by Peter Krulevitch, Dennis L. Polla, Mariam Maghribi, and Julie Hamilton for a Flexible Electrode Array for Artificial Vision published May 22, 2003 and U.S. Patent Application No. 2003/0097165 by Peter Krulevitch, Dennis L. Polla, Mariam Maghribi, Julie Hamilton, and Mark S. Humayun for a Flexible Electrode Array for Artificial Vision published May 22, 2003. U.S. Patent Applications Nos. 2003/0097166 and 2003/0097165 are incorporated herein in their entirety by this reference.

The electrodes 401A and 401B connect the electrode array 306 to the retina. The electrodes 401A and 401B stimulate the retina with a pattern of electrical pulses based on the sensed image signal. The system 300 receives the transmitted signal, derives power from the transmitted signal, decodes image data, and produces an electrical stimulus pattern at the retina based on the image data.

Implanting the electrode array 306 into the eye is a complex procedure. The system 300 provides a system for controlling movement of the electrode array 306 utilizing magnetic forces for implanting the electrode array 306 into the eye. The base of the electrode array 306 contains embedded or adhered to pieces 403. The pieces 403 in the polymer body portion 402A can be magnetic pieces or non-magnetic pieces.

In the surgical operation for implanting the retinal prosthesis, a system controls movement of the electrode array 306 through positioning of the base of the polymer body portion 402A utilizing magnetic forces. Systems for controlling movement of an implant utilizing magnetic forces are described in U.S. Pat. No. 6,475,233 issued Nov. 5, 2002 to Peter R. Werp et al for a method and apparatus for magnetically controlling motion direction of a mechanically pushed catheter and U.S. Patent Application No. 2004/0006301 by Jonathan C. Sell and Roger N. Hastings for a magnetically guided myocardial treatment system published Jan. 8, 2004. U.S. Pat. No. 6,475,233 and U.S. Patent Application No. 2004/0006301 are incorporated herein in their entirety by this reference.

The pieces 403 in the polymer body of the electrode array 306 and a system for controlling movement of the electrode array 306 utilizing magnetic forces guides the retinal implant to the desired locations against the retina. An external magnet in the system can be used to control movement of the electrode array 306 because of the pieces 403. The pieces 403 can be either magnetic pieces or non-magnetic pieces, or both. Once implanted, any magnetic pieces 403 can be removed if desired so that the magnetic material does not remain in the tissue.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A system for movement of an implant to a desired location, comprising:
a polymer body;
an electronic unit carried by said polymer body;
an electronic circuit in said polymer body operatively connected to said electronic unit;
pieces in said polymer body; and
means for controlling movement of said polymer body utilizing magnetic forces controlling movement of said pieces.

2. The system of claim 1 wherein said polymer body comprises a silicone body.

3. The system of claim 1 wherein said polymer body comprises a silicone body neural prosthesis.

4. The system of claim 1 wherein said polymer body comprises a silicone body cochlear prosthesis.

5. The system of claim 1 wherein said polymer body comprises a silicone body retinal prosthesis.

6. The system of claim 1 wherein said polymer body comprises a silicone body cortical prosthesis.

7. The system of claim 1 wherein said pieces in said polymer body comprise magnetic pieces.

8. The system of claim 1 wherein said pieces in said polymer body comprise non-magnetic pieces.

9. The system of claim 1 wherein said pieces in said polymer body comprise magnetic pieces and non-magnetic pieces.

10. The system of claim 1 wherein said polymer body comprises an elongated silicone body.

11. The system of claim 1 wherein said polymer body comprises an elongated silicone body and said electronic unit carried by said polymer body comprises an electrode.

12. The system of claim 1 wherein said polymer body comprises an elongated silicone body with a tip and wherein said pieces are located in said tip.

13. The system of claim 1 wherein said polymer body comprises an elongated silicone body with said electronic unit carried by said polymer body comprising electrodes embedded in said polymer body.

14. The system of claim 1 wherein said polymer body comprises an elongated silicone body and said electronic unit carried by said polymer body comprises electrodes embedded in said polymer body with said electronic circuit in said polymer body extending from said electronic unit along said elongated silicone body.

15. The system of claim 1 wherein said means for controlling movement of said polymer body utilizing magnetic forces controlling movement of said pieces includes a magnet.

16. The system of claim 1 wherein said means for controlling movement of said polymer body utilizing magnetic forces controlling movement of said pieces includes an electromagnet.

17. The system of claim 1 wherein said means for controlling movement of said polymer body utilizing magnetic forces controlling movement of said pieces includes a permanent magnet.

18. A method moving an implant to a desired location, comprising the steps of:
providing the implant with a polymer body;
providing an electronic unit that is carried by said polymer body;
providing an electronic circuit in said polymer body operatively connected to said electronic unit;
providing pieces in said polymer body; and
using magnetic forces on said pieces to move the implant to the desired location.

19. The method moving an implant to a desired location of claim 18 wherein said step of using magnetic forces on said pieces to move the implant to the desired location comprises using an electromagnet on said pieces to move the implant to the desired location.

20. The method moving an implant to a desired location of claim 18 wherein said step of using magnetic forces on said pieces to move the implant to the desired location comprises using a permanent magnet on said pieces to move the implant to the desired location.

21. The method moving an implant to a desired location of claim 18 wherein said step of providing pieces comprises providing nonmagnetic pieces and said step of using magnetic forces on said pieces to move the implant to the desired location comprises using an electromagnet on said nonmagnetic pieces to move the implant to the desired location.

22. The method moving an implant to a desired location of claim 18 wherein said step of providing pieces comprises providing nonmagnetic pieces and said step of using magnetic forces on said pieces to move the implant to the desired location comprises using a permanent magnet on said nonmagnetic pieces to move the implant to the desired location.

23. The method moving an implant to a desired location of claim 18 wherein said step of providing pieces comprises providing magnetic pieces and said step of using magnetic forces on said pieces to move the implant to the desired location comprises using an electromagnet on said magnetic pieces to move the implant to the desired location.

24. The method moving an implant to a desired location of claim 18 wherein said step of providing pieces comprises providing magnetic pieces and said step of using magnetic forces on said pieces to move the implant to the desired location comprises using a permanent magnet on said magnetic pieces to move the implant to the desired location.

25. A method moving an implant, comprising the steps of:
providing the implant with a polymer body;
providing an electronic unit that is carried by said polymer body;
providing an electronic circuit in said polymer body operatively connected to said electronic unit;
providing pieces in said polymer body; and
using magnetic forces on said pieces to move the implant.

26. The method moving an implant of claim 25 wherein said step of using magnetic forces on said pieces to move the implant comprises moving the implant to a desired location.

27. The method moving an implant of claim 25 wherein said step of using magnetic forces on said pieces to move the implant comprises moving the implant from an existing location.

28. The method moving an implant of claim 25 wherein said step of using magnetic forces on said pieces to move the implant comprises using magnetic forces on said pieces to move the implant to a location and using magnetic forces on said pieces to move the implant from the location.
